(19) United States
(12) Patent Application Publication
(10) Pub. No.: US 2026/0123941 A1
(43) Pub. Date: May 7, 2026

(54) SPINAL DECORTICATION AND GRAFTING

(71) Applicant: PROVIDENCE MEDICAL TECHNOLOGY, INC., Pleasanton, CA (US)

(72) Inventors: Christopher U. Phan, Dublin, CA (US); Joseph O'Brien, Bethesda, MD (US); Eric Feuchtbaum, Arlington, VA (US)

(21) Appl. No.: 19/119,927

(22) PCT Filed: Oct. 11, 2023
(86) PCT No.: PCT/US2023/034880

§ 371 Date: Apr. 10, 2025

Related U.S. Application Data
(60) Provisional application No. 63/415,093, filed on Oct. 11, 2022.
(60) Provisional application No. 63/449,126, filed on Mar. 1, 2023.

(51) Int. Cl. A61B 17/16; A61B 17/00; A61B 17/34; A61F 2/46

(52) U.S. Cl. CPC A61B 17/1671; A61B 17/3423; A61F 2/4601; A61B 2017/0046; A61B 2017/3488

(57) ABSTRACT

A decortication and grafting device for lumbar fusion procedures includes a distal decortication tip and a tubular portion extending to a proximal handle. The tubular portion is configured to receive and allow passage of a bone graft material therethrough. A graft injection device coupled to the proximal handle of the decortication and grafting device injects graft material into the tubular portion which is then deposited on a bony surface decorticated by the decortication tip. A system for decortication and grafting further includes an access portal device defining a first lumen configured to slidably couple to an anchor device, such as a k-wire, and a second lumen configured to accommodate passage of various surgical instruments, such as a dilation device and the decortication and grafting device, and guide the instruments to a targeted surgical site.

### CROSS-REFERENCE TO RELATED APPLICATIONS

This application claims the benefit of U.S. Provisional Application No. 63/415,093, filed Oct. 11, 2022, the disclosure of which is hereby incorporated by reference in its entirety.

This application also claims the benefit of U.S. Provisional Application No. 63/449,126, filed Mar. 1, 2023, the disclosure of which is hereby incorporated by reference in its entirety.

### TECHNICAL FIELD

This disclosure is directed to medical devices, systems and methods. More specifically, this disclosure is directed to devices, systems and methods related to spinal fusion procedures involving bone decortication and grafting.

### BACKGROUND

Chronic back problems are one of the most common causes of pain and disability in the United States and other developed countries. Adverse spinal conditions may be characteristic of age. Conditions such as Degenerative Disc Disease (DDD) and spinal stenosis can result in a reduction of foraminal area (i.e., the available space for the passage of nerves and blood vessels), which may compress nerve roots and cause radicular pain. Spinal fusion, in which two adjacent vertebrae are fused together using plates, screws and other implants, is often performed to increase space (“spinal distraction”) and reduce motion between adjacent vertebrae, thereby preventing impingement of the spinal cord and nerve roots passing through openings in the vertebral column.

One of the reasons that spinal fusion surgery is typically very invasive is that, due to the position of the spinal cord in back of (posterior to) the central vertebral bodies of the spine, many of the procedures require entering the patient through the front of the body (an anterior approach) and dissecting through various tissues to gain access to the spine. Fusion procedures performed on the lumbar spine, for instance, often require dissecting through the abdomen. Unfortunately, most techniques and devices used for performing spinal fusion are relatively invasive, high-risk, and necessitate difficult recovery and rehabilitation.

Accordingly, a need exists for new, less invasive devices and methods for vertebral fusion.

### SUMMARY

The present disclosure describes devices, systems and associated methods for spinal decortication and grafting. In accordance with embodiments of the present disclosure, a spinal decortication and grafting system may include an access portal device defining a first lumen and a second lumen, each lumen extending longitudinally through a length of the access portal device. The first lumen may be configured to receive an anchoring device and the second lumen may be configured to receive one or more surgical instruments. The system may also include a decortication device having a tubular portion and a distal decortication member. A graft injection device configured to couple with and push grafting material through the tubular portion of the decortication device may also be included. The tubular portion of the decortication device may be configured to extend into and through the second lumen of the access portal device until the decortication member is positioned at a targeted bony surface.

In some examples, the anchoring device includes a k-wire. In some examples, the anchoring device includes an access chisel. In some examples, the cross-sectional dimensions of the second lumen are greater than the cross-sectional dimensions of the first lumen. In some examples, the graft injection device may include a plunge member and a tubular portion defining an inner lumen, the plunge member configured to urge the graft material through the inner lumen in response to manual actuation. In some examples, the decortication device further comprises a handle attached to a proximal end of the tubular portion. In some examples, the handle defines a coupling component configured to couple to a distal end portion of the graft injection device. In some examples, the coupling component includes a luer-thread fitting. In some examples, the distal decortication member includes a plurality of serrated teeth or ridges configured to decorticate the targeted bony surface via rotation or scraping. In some examples, the distal decortication member includes a substantially spherical burr covered in wavy ridges, serrated teeth or protrusions. In some examples, the system may also include a dilation device configured to extend through the second lumen, the dilation device including a tapered distal portion configured to spread apart tissue at or near the targeted bony surface. In some examples, the access portal device is configured to accommodate differently sized decortication devices having differently configured distal decortication members. In some examples, the targeted bony surface is a feature of a lumbar vertebrae.

In accordance with embodiments of the present disclosure, a decortication device for spinal fusion procedures includes an elongate tubular portion configured to receive and facilitate passage of a graft material therethrough, a decortication member attached to a distal end of the elongate tubular portion and configured to decorticate a bony surface, as well as a handle member attached to a proximal end of the elongate tubular portion and configured to rotate the decortication member in response to manual actuation.

In some examples, the handle member defines a coupling component configured to couple to a graft injection device and receive the graft material ejected therefrom. In some examples, the coupling component includes a luer-thread fitting. In some examples, the decortication member includes a plurality of serrated teeth or ridges configured to decorticate the bony surface via rotation or scraping. In some examples, the decortication device may also include an adjustable depth stop on the tubular portion configured to prevent over-extension of the decortication member beyond the bony surface. In some examples, the handle member includes an angled body such that a coupling component defined by the handle member is angled with respect to a longitudinal axis of the elongate tubular portion. In some examples, a distal portion of the elongate tubular portion may be configured to curve or contort away from a longitudinal axis of the elongate tubular portion. In some examples, the decortication device also includes an inner stylet extendable through the elongate tubular portion. In some examples, the bony surface is a feature of a lumbar vertebrae

In accordance with some embodiments of the present disclosure, a method of decorticating a bony surface and depositing a graft material thereon may be implemented using a system and/or device according to any of the aforementioned examples. In some examples, the method is performed in connection with a lumbar spinal fusion procedure

### BRIEF DESCRIPTION OF THE DRAWINGS

FIG. 1 is a perspective view of a decortication device and Detail A is close-up view of a distal tip portion thereof in accordance with embodiments of the present disclosure.

FIG. 2A is a perspective view of an access portal device in accordance with embodiments of the present disclosure.

FIG. 2B is a perspective view of the access portal device shown in FIG. 2A coupled with a dilation device in accordance with embodiments of the present disclosure.

FIG. 2C is a perspective view of the dilation device shown in FIG. 2B being inserted into or withdrawn from the access portal device of FIGS. 2A and 2B in accordance with embodiments of the present disclosure.

FIG. 3 is a perspective view of the decortication device of FIG. 1 coupled to a graft injection device in accordance with embodiments disclosed herein.

FIG. 4A is snapshot of a step performed during a decortication and grafting procedure in accordance with embodiments of the present disclosure.

FIG. 4B is a snapshot of another step performed during a decortication and grafting procedure in accordance with embodiments of the present disclosure.

FIG. 4C is a snapshot of another step performed during a decortication and grafting procedure in accordance with embodiments of the present disclosure.

FIG. 4D is a snapshot of another step performed during a decortication and grafting procedure in accordance with embodiments of the present disclosure.

FIG. 4E is a snapshot of another step performed during a decortication and grafting procedure in accordance with embodiments of the present disclosure.

FIG. 4F is a snapshot of another step performed during a decortication and grafting procedure in accordance with embodiments of the present disclosure.

FIG. 4G is a snapshot of another step performed during a decortication and grafting procedure in accordance with embodiments of the present disclosure.

FIG. 4H is a snapshot of another step performed during a decortication and grafting procedure in accordance with embodiments of the present disclosure.

FIG. 5 is a perspective view of a kit comprised of devices for performing a decortication and grafting procedure in accordance with embodiments of the present disclosure.

FIG. 6A is a perspective view of an access portal device anchored at a targeted surgical location in accordance with embodiments of the present disclosure.

FIG. 6B is a close-up view of a distal end portion of the access portal device shown in FIG. 6A in accordance with an embodiment of the present disclosure.

FIG. 6C is a close-up view of a distal end portion of the access portal device shown in FIG. 6A in accordance with another embodiment of the present disclosure

FIG. 7 is a perspective view of an access portal device anchored at a targeted surgical location in accordance with embodiments of the present disclosure.

FIG. 8 is a front view of a graft injection device in accordance with embodiments of the present disclosure.

FIG. 9A is a perspective view of a decortication device coupled to a graft injection device in accordance with embodiments of the present disclosure.

FIG. 9B is a perspective view of a distal end portion of the decortication device shown in FIG. 9A in accordance with an embodiment of the present disclosure.

FIG. 9C is a perspective view of a distal end portion of the decortication device shown in FIG. 9A in accordance with another embodiment of the present disclosure.

FIG. 10A is a perspective view of a steerable graft delivery device coupled to a graft injection device in accordance with embodiments of the present disclosure.

FIG. 10B is a perspective view of a distal end portion of the steerable graft delivery device shown in FIG. 10A during its deployment near a target site in accordance with embodiments of the present disclosure.

FIG. 11A is a perspective view of an access portal device coupled to a dilation device in accordance with embodiments of the present disclosure.

FIG. 11B is a perspective view of the dilation device shown in FIG. 11A being inserted into or withdrawn from the access portal device of FIG. 11A in accordance with embodiments of the present disclosure.

FIG. 11C is a perspective view of the access portal device shown in FIGS. 11A and 11B in accordance with embodiments of the present disclosure.

FIG. 12 is a perspective view of an access portal device that includes a pedicle stylet in accordance with embodiments of the present disclosure.

FIG. 13A is a perspective view of a retractor portal in a first configuration in accordance with embodiments of the present disclosure.

FIG. 13B is a perspective view of the retractor portal shown in FIG. 13A in a second configuration in accordance with embodiments of the present disclosure.

FIG. 13C is a perspective view of the retractor portal shown in FIG. 13A in a third configuration in accordance with embodiments of the present disclosure.

FIG. 14 includes perspective views of a decortication device and components thereof in accordance with embodiments of the present disclosure.

FIG. 15 includes perspective views of various decortication tips in accordance with embodiments of the present disclosure.

FIG. 16 is a perspective view of two access portal devices and two decortication devices in accordance with embodiments of the present disclosure.

FIG. 17A is a depiction of a step performed during a decortication and grafting procedure in accordance with embodiments of the present disclosure.

FIG. 17B is a depiction of another step performed during a decortication and grafting procedure in accordance with embodiments of the present disclosure.

FIG. 17C is a depiction of another step performed during a decortication and grafting procedure in accordance with embodiments of the present disclosure.

FIG. 17D is a depiction of another step performed during a decortication and grafting procedure in accordance with embodiments of the present disclosure.

FIG. 17E is a depiction of another step performed during a decortication and grafting procedure in accordance with embodiments of the present disclosure.

FIG. 17F is a depiction of another step performed during a decortication and grafting procedure in accordance with embodiments of the present disclosure.

### DETAILED DESCRIPTION

The following description of certain embodiments is merely exemplary in nature and is in no way intended to limit the invention or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

As used herein, “vertebrae” may refer to lumbar vertebrae unless otherwise indicated. Embodiments are not limited to lumbar vertebrae, however, and may encompass other vertebrae or groups of vertebrae along the spine of a subject.

As used herein, “subject” may refer to a human (e.g., a patient) afflicted with a spinal condition, e.g., spinal stenosis, injury or disease, which may be degenerative. The condition may impact the integrity and/or positioning of one or more vertebrae, which may cause chronic pain and instability.

As used herein, “device” may refer to a spinal fusion device or component thereof configured for decorticating, grafting and/or fusing at least a portion of one or more vertebrae.

The terms “distal” and “proximal” are used to refer to a position or direction relative to the treating clinician, such as a surgeon. “Distal” and “distally” refer to a position that is distant from, or in a direction away from, the treating clinician. “Proximal” and “proximally” refer to a position that is near, or in a direction toward, the treating clinician. The terms “posterior” and “anterior” refer to the back and front, respectively, of the body of a subject.

As used herein, “decortication” refers to the process by which an outer surface of a bone is roughened or removed and the underlying cancellous bone exposed. Relative to the outer bone surface, the cancellous bone more effectively retains deposited graft material, thereby promoting healing and bone growth after surgery.

The terms “device” and “instrument” may be used interchangeably herein. The terms “component,” “member” and “tool” may also be used interchangeably.

For the following defined terms, certain definitions shall be applied unless a different definition is given elsewhere in this disclosure. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether or not explicitly indicated. The term “about” generally refers to a range of numbers that one of skill in the art would consider equivalent to the recited value (e.g., having the same function or result). In many instances, the term “about” can include numbers that are rounded to the nearest significant figure. The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.).

Posterior lumbar decortication and grafting is an aspect of lumbar spinal fusion procedures, during which a surgeon decorticates and deposits graft material along one or more bony surfaces of various features of the lumbar vertebrae, non-limiting examples of which include the lumbar facet joint(s), pedicle gutter and transverse process, to ultimately achieve posterior fusion after a healing period. Decortication and grafting are typically preceded by a pedicle screw and rod (PSR) procedure to fixate the lumbar spine. With minimally invasive lumbar PSR surgery gaining popularity over traditional open lumbar PSR surgery, it has become a challenge to properly decorticate and lay graft in these areas without further disrupting the incision size (making it larger) created during posterior entry. The lack of options for decortication and grafting in a minimally invasive PSR procedure may compromise clinical outcomes by inadequately decorticating and laying graft to the areas of the posterior lumbar bony anatomy, and/or by increasing by the incision size required to adequately decorticate and lay graft to these areas.

Devices, systems and methods for performing minimally invasive posterior fusion procedures involving decortication and grafting of the lumbar spine are disclosed herein. The fusion procedures may be performed in critical areas, including the facet joint, pedicle gutter and transverse process, at least one of which may be accessed, decorticated and grafted using one or more of the disclosed devices. Embodiments may utilize an anchor device (e.g., a k-wire) to secure an access portal device, such as a dual-lumen access portal device, to a targeted bony surface of a vertebral feature, such as the pedicle, prior to pedicle screw placement. The anchor member(s) may be extended through a first lumen of the access portal device to secure the device in position, while a second lumen provides a stable pathway to the surgical site for additional device(s) configured to dilate tissue, decorticate bone and/or lay graft material. By anchoring the access portal device to a bony surface (e.g., pedicle) via a lumen defined by the portal, the disclosed access portal device may eliminate the need to anchor a portal device to a table or bed for stabilization, as is common with preexisting approaches. Radiopaque markers may be included at or near the distal end of the access portal device to provide position points visible under fluoroscopic imaging to guide and orient the portal over the desired surgical site. Notably, the access portal device may be shaped and sized to avoid increasing or disrupting the typical incision size needed for minimally invasive pedicle screw and rod placement.

After portal anchoring, a single device for both decortication and graft delivery (hereinafter the “decortication device”) may be deployed through the second lumen of the access portal device, which stabilizes the decortication device for precise operation. The decortication device may include an elongate tubular portion for graft material delivery, along with a distal end having serrated teeth or other protruding features for bone decortication. The inner lumen defined by the tubular portion is configured to receive and facilitate delivery of graft material in a distal direction toward the targeted surgical location after/during decortication of the same via the distal end of the device. The decortication device may also include a proximal handle that defines a coupling component (e.g., luer-thread fitting) configured for attachment to a graft injection device (e.g., syringe) loaded with graft material. Accordingly, the decortication device may be configured for integrated bone decortication and graft delivery, which may be performed simultaneously or in quick succession.

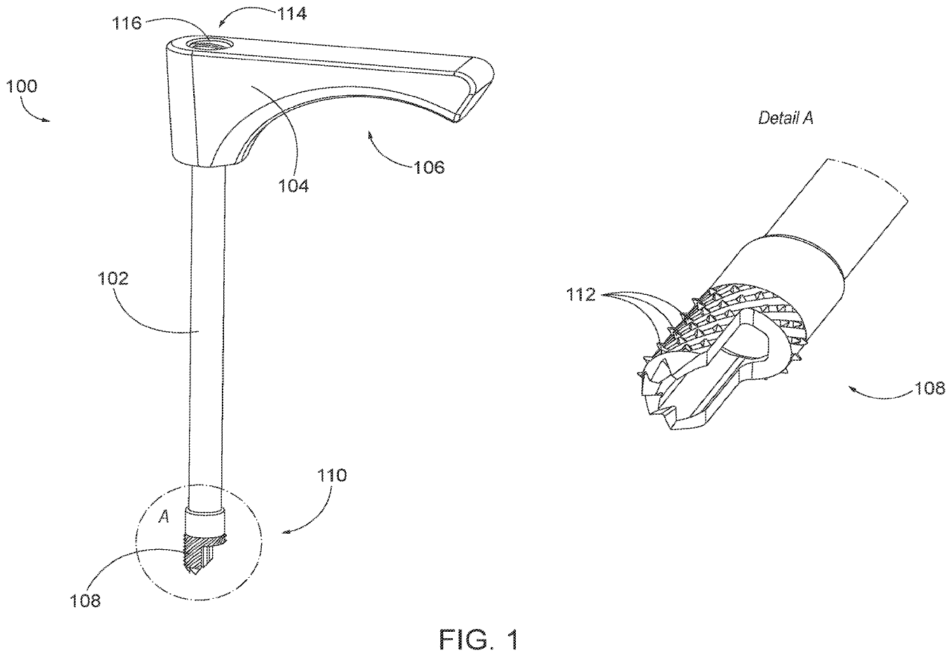

An embodiment of the decortication device 100 is shown in FIG. 1. The decortication device 100 includes a tubular portion 102 attached, fixed or otherwise coupled to a handle 104 at a proximal end portion 106 of the device. A decortication component, head or tip 108 is included at a distal end portion 110 of the device 100. The decortication tip 108 may include or be defined by a textured surface, whereupon the decortication tip 110 is moved to decorticate one or more bony surfaces. The decortication tip 108 may include a variety of configurations and decortication features. For example, the decortication tip 108 may have a rough surface defined by a plurality of teeth or projections 112 configured to decorticate bone when pressed, rotated or otherwise scraped against a bony surface. In operation, a user may manipulate (e.g., rotate clockwise/counterclockwise) the decortication device 100 via the handle 104 and/or tubular portion 102 to decorticate a targeted bone using the decortication tip 108.

As further shown, the decortication device 100 may include a coupling feature, mechanism or component 114 configured to couple the device with a graft injection device. The coupling component 114 of the illustrated embodiment comprises a luer-thread fitting 116. Non-limiting examples of coupling components utilized in different embodiments may include snap-fit or lock-and-key mechanisms.

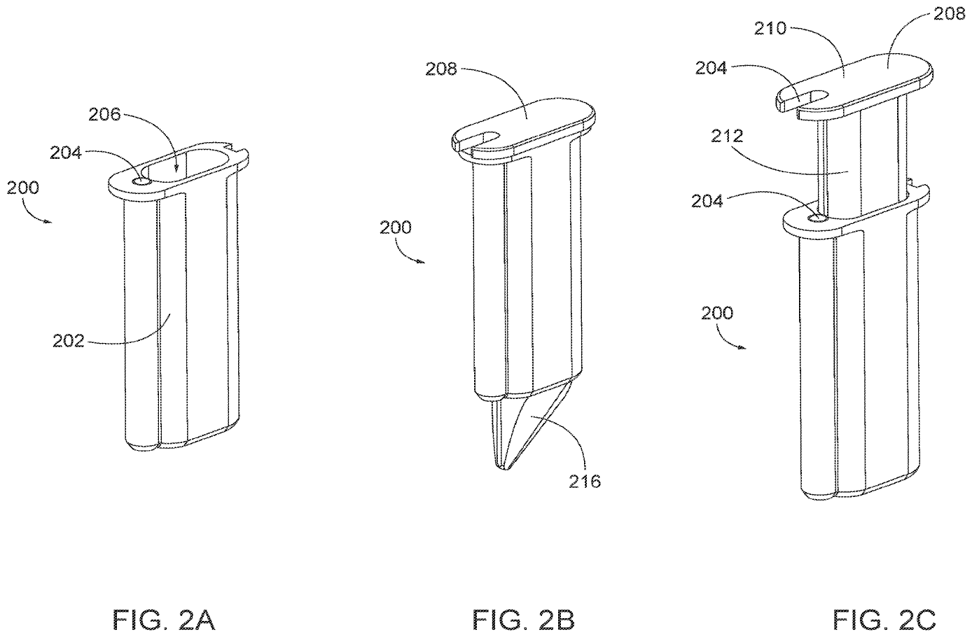

The lumen defined by the tubular portion 102 may be sized and configured to receive and facilitate passage of a graft material therethrough, from the proximal end 106 to the distal end 110 of the device 100, where the graft material exits for deposition on a decorticated bony surface. To ensure precise decortication and graft delivery, the decortication device 100 may be inserted through an access portal device, an example of which is shown in FIGS. 2A-2C. The access portal device 200 includes a body 202 defining a first lumen 204 and a second lumen 206. The first lumen 204 may be sized and configured to accommodate the receipt and passage of one or more anchor devices therethrough, non-limiting examples of which may include a k-wire. The second lumen 206 may be configured to accommodate the receipt and passage of one or more devices and materials used during a spinal fusion procedure, non-limiting examples of which may include the decortication device 100, stylets, BMP, OP1, silicone, PMMA bone cement, or hydrogel. While both lumens extend longitudinally through the entire length of the access portal device 200, the dimensions of the second lumen 206 may be larger than the first.

As further shown, the access portal device 200 may include or be coupled with a tissue dilation device 208 (or “dilator”) configured to spread apart tissues surrounding or posterior to the targeted bones, thereby facilitating access to the surgical site. The dilation device 208 may include a cap 210 and a shaft portion 212, the shaft portion configured to slide proximally and distally through the second lumen 206. The cap 210 may define a cutout portion 214 sized and shaped to avoid covering or blocking the first lumen 204. The distal end portion 216 of the dilation device 208 may be configured to be urged through or between tissues and spread the tissues apart to enable clear access to the adjacent or underlying bony surfaces. The distal end portion 216 may be tapered to facilitate smooth, gradual penetration and stretching of the tissues.

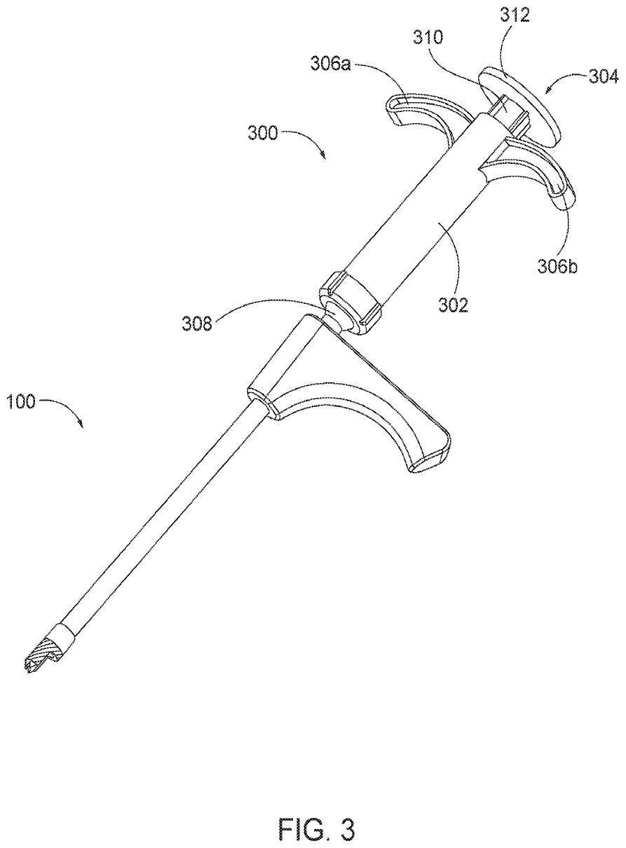

FIG. 3 shows the decortication device 100 coupled with a graft injection device 300. The graft injection device 300 may comprise or resemble a syringe, which may include a barrel or tubular body 302 defining an inner lumen, a plunge member 304, and one or more user engagement members 306a, b. A distal end portion 308 of the graft injection device 300 may define a funnel culminating in a distal opening configured to release graft material into the coupling component 116 and tubular portion 102 of the decortication device 100. The plunge member 304 may include a rod or shaft member 310 resembling a piston that is coupled, attached or formed integrally with a proximal handle or plunge cap 312. The diameter of the shaft member 310 may be slightly smaller than the inner diameter of the lumen defined by the tubular body 302, such that the shaft member 310 may be slid within the body 302 to urge graft material therethrough.

The tubular body 302 may be filled or substantially filled with a graft material, which may be allogenic, autogenous, or synthetic. Embodiments of the graft injection device 300 may be configured to deliver various amounts of graft material, including pre-set, defined amounts provided in distinct aliquots or boluses. The graft injection device 300 may be loaded with the graft material directly or via removable cartridges containing the material. In some examples, the plunge cap 312 may define a proximal aperture configured to receive the graft material. After secure placement of the decortication device 100 at a surgical site, the graft injection device 300 may be coupled thereto and the graft material expelled from the injection device via distal sliding of the shaft member 310. Distal movement of the shaft member 310 may be driven by user actuation of the plunge member 304, which may involve pressing the plunge cap 312 into the tubular body 302 while gripping or holding the user engagement members 306a, b with two or more fingers. In the illustrated embodiment, the engagement members 306a, b comprise arcuate handles extending from opposite sides of the tubular body 302, but the configuration of the engagement members 306a, b may vary.

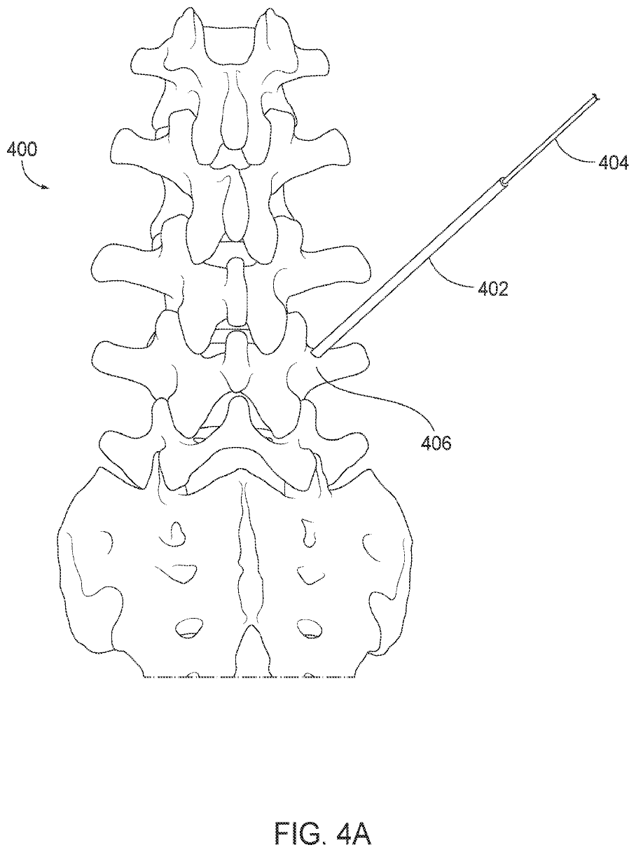

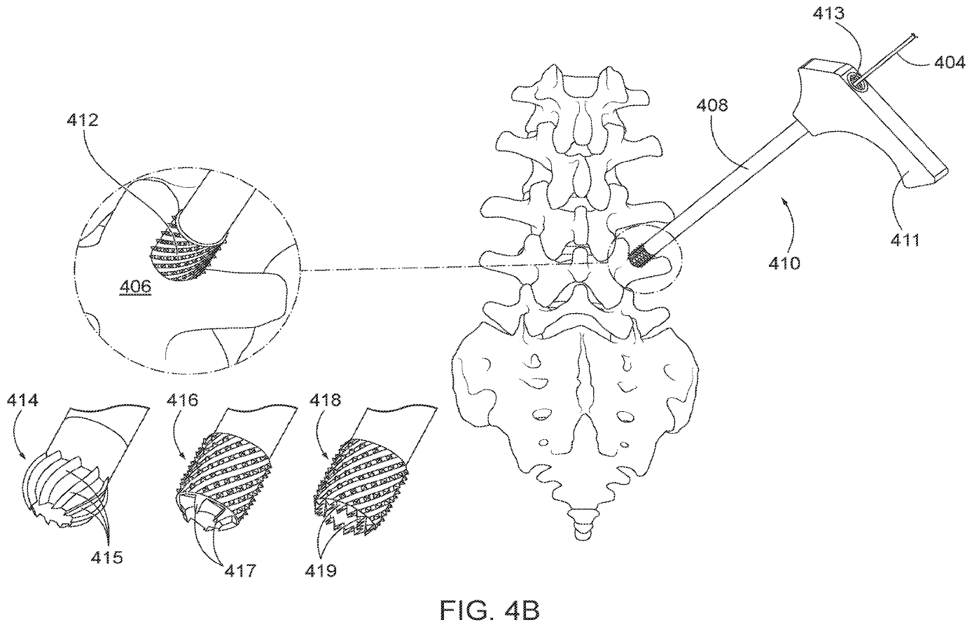

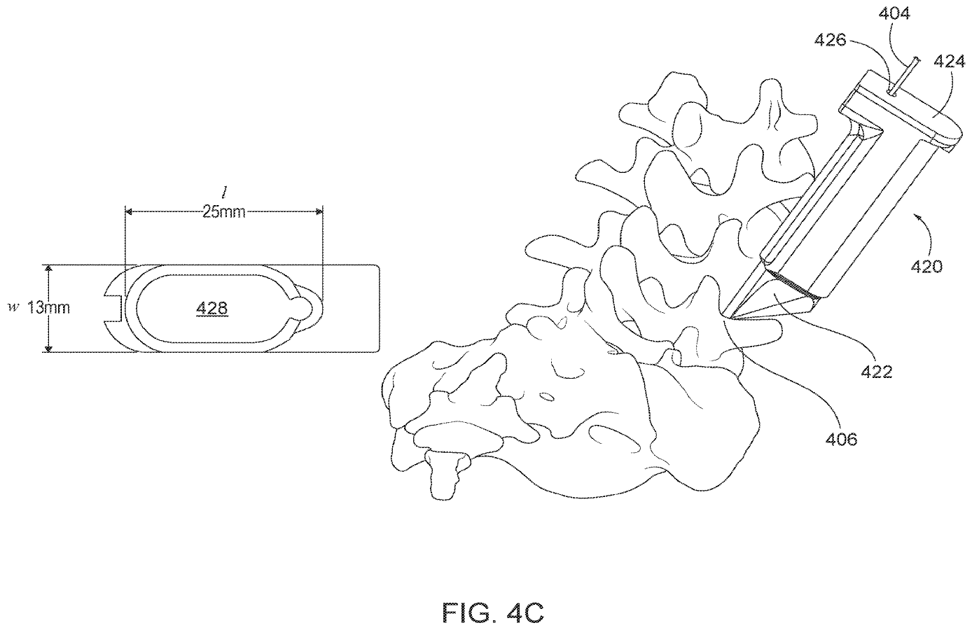

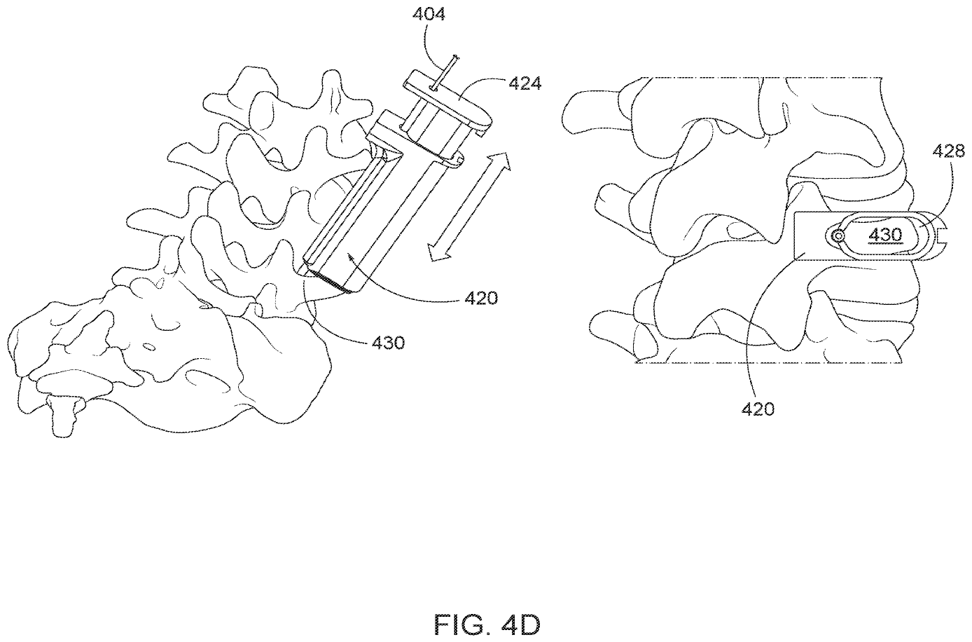

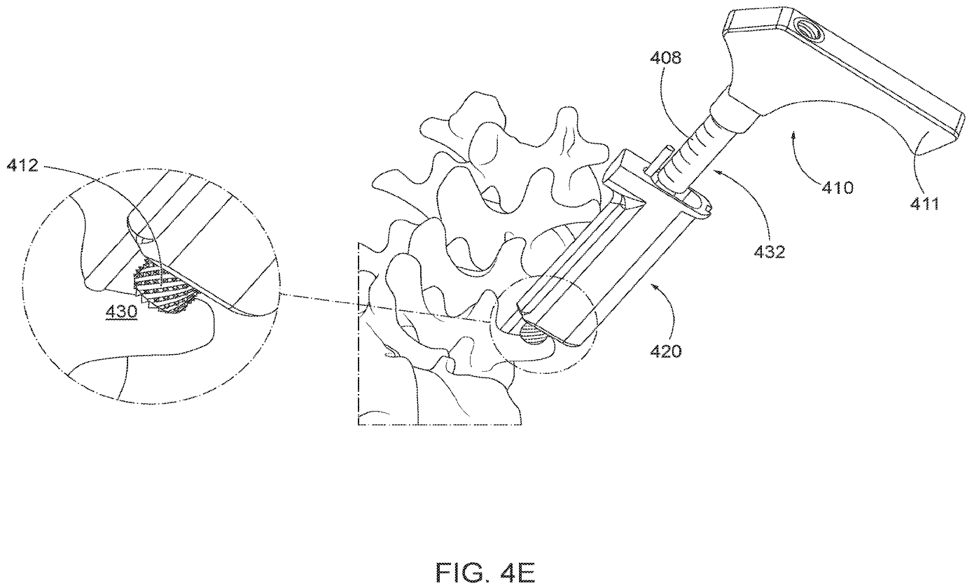

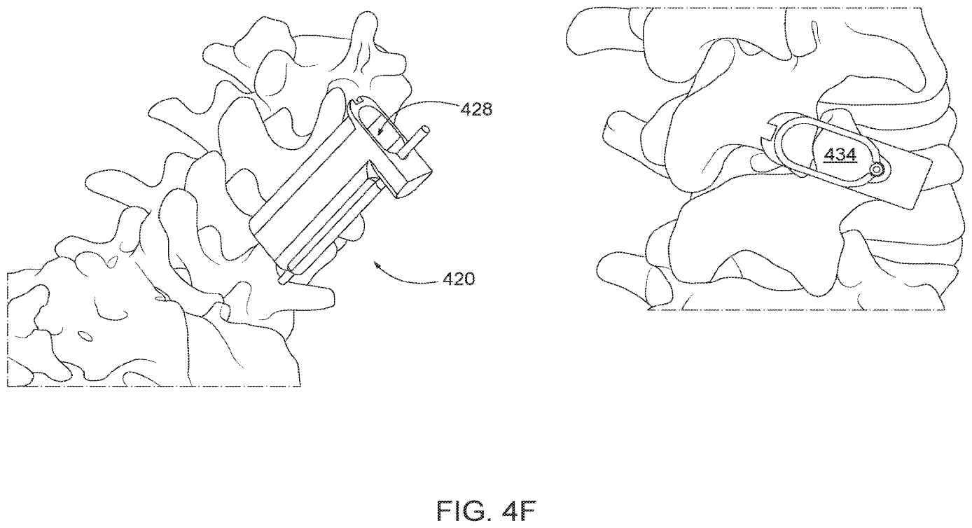

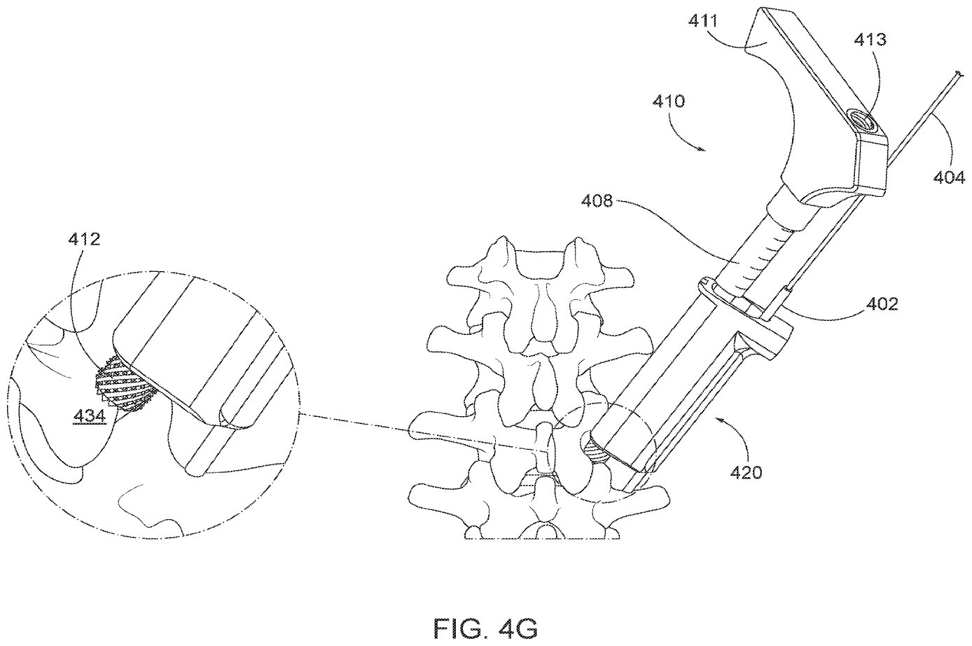

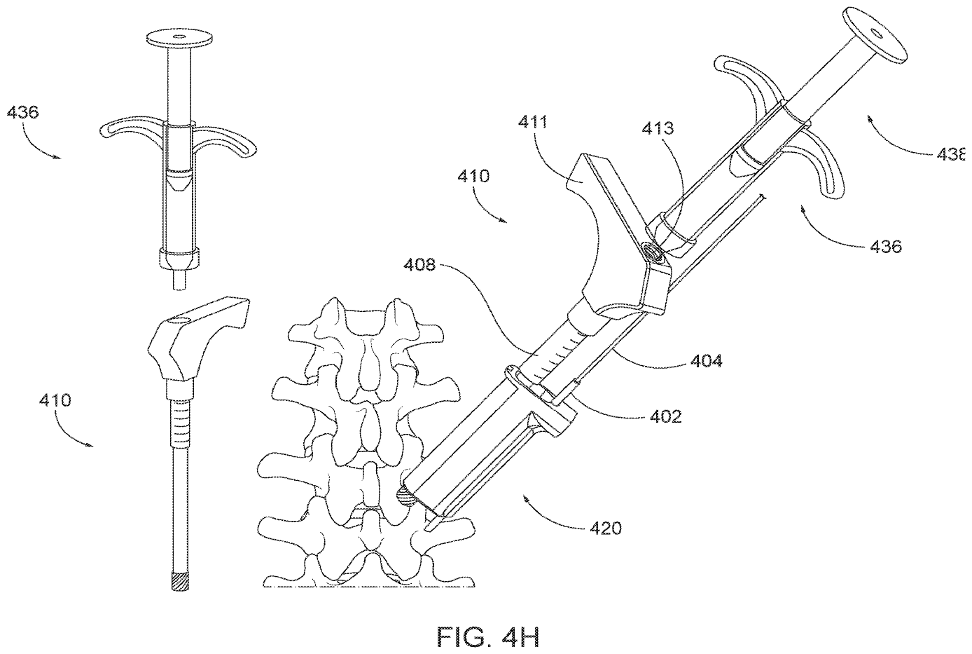

FIG. 4A is an illustration of an initial step of a decortication and grafting procedure performed in a lumbar spinal region 400 in accordance with embodiments disclosed herein. As shown, the procedure may involve sliding an anchor device 402 over a k-wire 404 until the distal end of the anchor device 402 is positioned at a bony surface 406 targeted for decortication, which in the illustrated embodiment comprises a pedicle gutter. The size, configuration and/or type of anchor device utilized may vary. The particular anchor device 402 depicted in FIG. 4A is an 11 gauge (3 mm) anchor.

Subsequently, the procedure may involve sliding the tubular portion 408 of a decortication device 410 over the k-wire 404 and anchor device 402 until the decortication tip 412 is positioned against the bony surface 406 (FIG. 4B). The decortication tip 412, positioned opposite the proximal handle 411 and coupling component 413 of the decortication device 410, may then be rotated, slid or otherwise moved or scraped against the bony surface 406 to decorticate the surface. The configuration of the decortication tip 412 may vary. Decortication tip 412 comprises a spiral pattern of serrations; other embodiments may include additional/alternative features. Decortication tip 414, for example, includes a decortication burr having a generally round or spherical shape covered in wavy, arcuate ridges 415. Decortication tip 416 comprises a spiral pattern of serrations and a plurality of distally extending prongs 417. Decortication tip 418 comprises a spiral pattern of serrations and a plurality of distally extending ridges or pointed teeth 419.

An access portal device 420 may be slid over the anchor device 402 and k-wire 404, led by a tapered distal end portion 422 of a dilation device 424, until the distal end portion 422 reaches the decorticated bony surface 406 (FIG. 4C). Precise positioning of the access portal device 420 may be achieved by advancing the access portal device 420 over the anchor device 402 and k-wire 404 via a first lumen 426 defined by the access portal device 420. The cross-sectional dimensions of a second lumen 428 of the access portal device 420 (perpendicular to the longitudinal axis) may vary to accommodate one or more instruments used for decortication and grafting procedure. In the embodiment shown, the cross-sectional length l of the second lumen 428 is about 25 mm and the width w is about 13 mm. Other embodiments of the second lumen 428 may be sized and shaped differently. For example, the cross-sectional length of the second lumen 428 may range from about 10 mm to about 50 mm, and the cross-sectional width may range from about 5 mm to about 30 mm. Embodiments may also feature a lumen 428 having a substantially circular cross-sectional shape.

Sliding the access portal device 420 down the anchor device 402 and k-wire 404 may position the distal opening of the second lumen 428 adjacent to the bony surface 406, such that the distal opening is positioned over a second bony feature or surface 430, which may comprise the transverse process. Some manipulation of the access portal device 420 may be necessary to position the distal opening precisely over the second bony surface 430, after which the outer sleeve or body of the access portal device 420 may be pushed down and the dilation device 424 removed proximally (FIG. 4D).

The decortication device 100 may then be inserted into and distally through the second lumen 428 of the access portal device (FIG. 4E). A depth stop 432 included on the tubular portion 408 of the decortication device 410 may be adjusted to a desired depth, and the second bony surface 430 decorticated by rotating the proximal handle 411 clockwise/counterclockwise.

The access portal device 420 may then be repositioned (e.g., rotated) to position the distal opening of the second lumen 428 over a third bony feature or surface 434, which may comprise a facet joint (FIG. 4F). The decortication device 410 may be re-inserted into and through the second lumen 428 of the access portal device 420, the depth stop 432 adjusted as necessary, and a portion of or adjacent to the third bony surface 434 (e.g., the transverse process) decorticated via clockwise/counterclockwise rotation the proximal handle 411 (FIG. 4G).

As shown in FIG. 4H, a graft injection device 436 may be attached to the coupling component 413 of the proximal handle 411 of the decortication device 410. Graft material, e.g., allograft, contained within the graft injection device 436 may then be injected into the decorticated areas via the decortication device (FIG. 4H). The plunge member 438 of the injection device 436 may be pushed distally to dispense any remaining graft material out of the tubular portion 408 of the decortication device 410 (FIG. 4I).

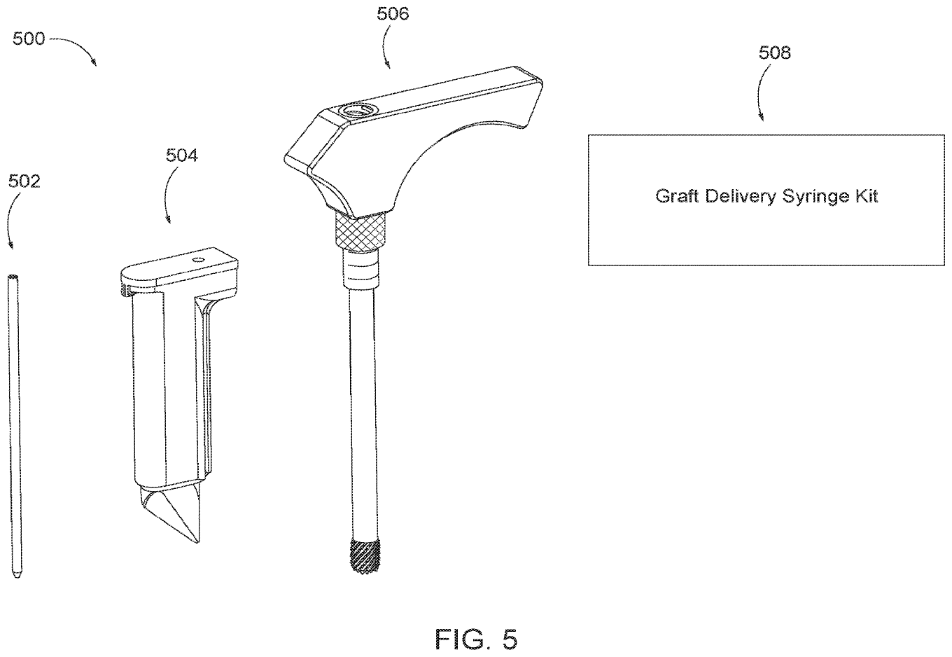

FIG. 5 shows a decortication and grafting kit 500 provided in accordance with embodiments disclosed herein. Examples of the kit 500 may include a pedicle anchor 502, an access portal device 504, a decortication device 506, and graft delivery syringe kit 508. Allograft may also be included with the kit 500.

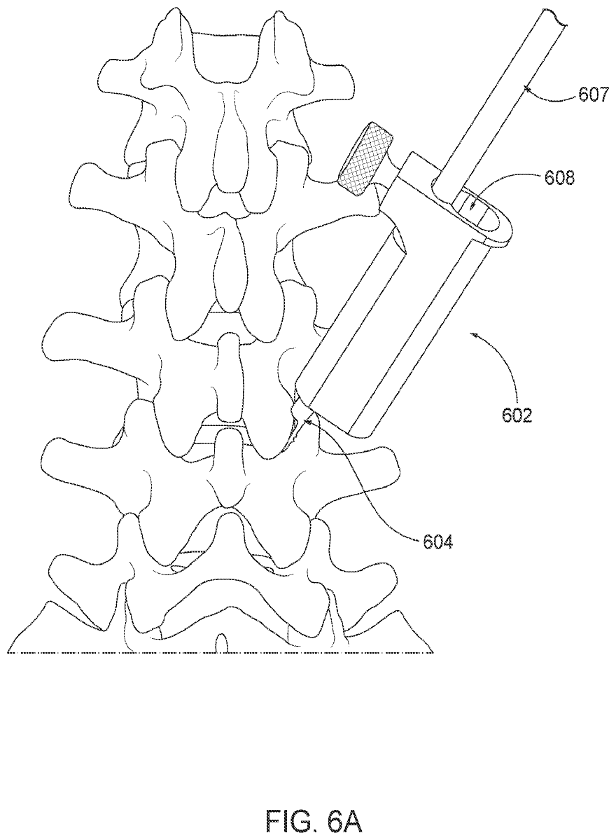

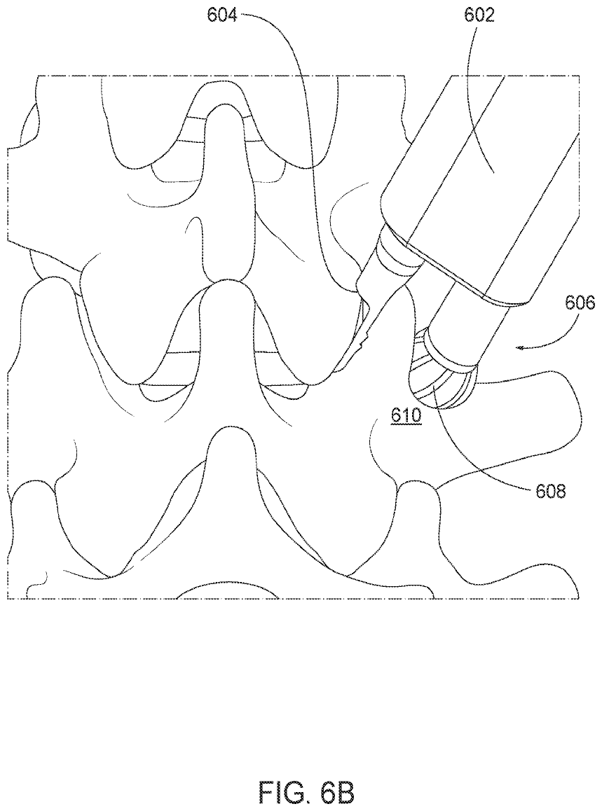

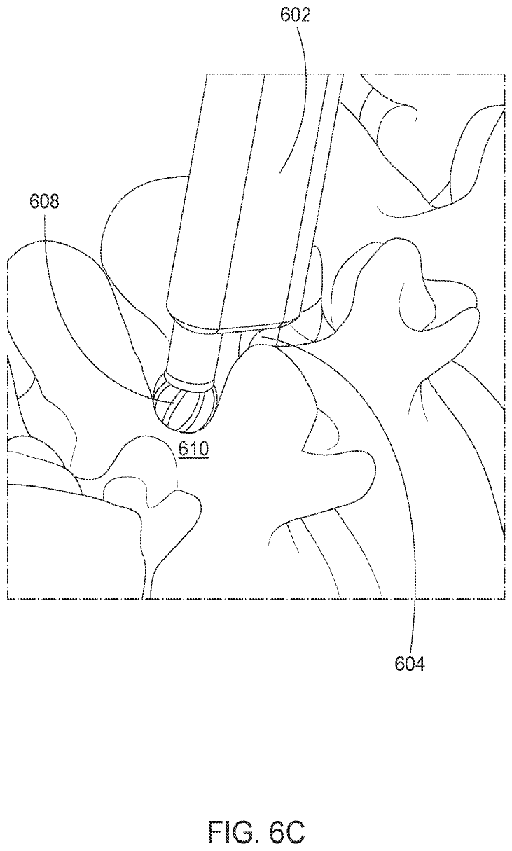

FIG. 6A shows an embodiment of an access portal device 602 and a facet access chisel 604 protruding from a distal end of the device 602 and attached or formed with an elongate anchoring shaft 607. As further shown in FIG. 6B, the facet access chisel 604 can be inserted or wedged in a facet to guide and anchor the device 602 at the target site. A decortication device 606 can be slid distally through a lumen 608 defined by the access portal device 602 until the decortication tip 608 is positioned at the targeted grafting site 610.

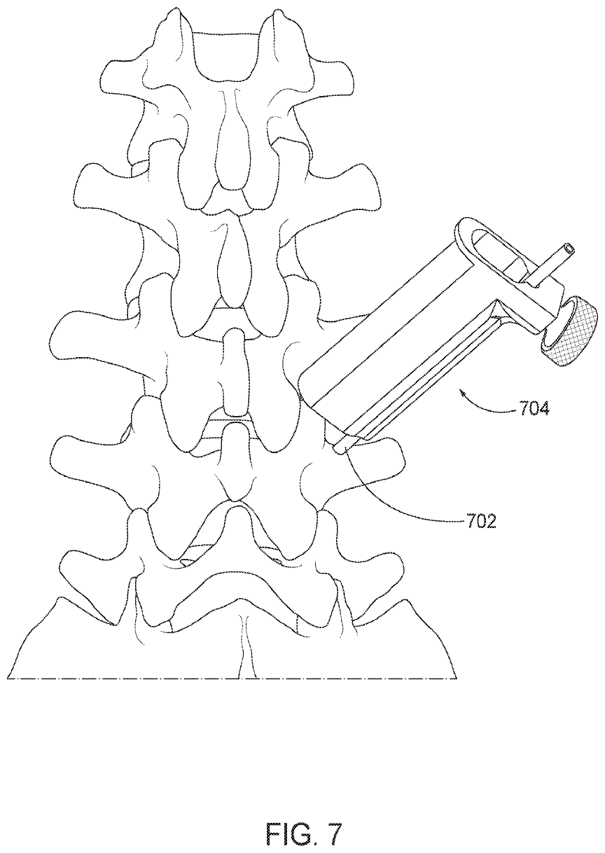

FIG. 7 depicts an embodiment of a disclosed system featuring a pedicle k-wire 702 as an anchoring point for an access portal device 704.

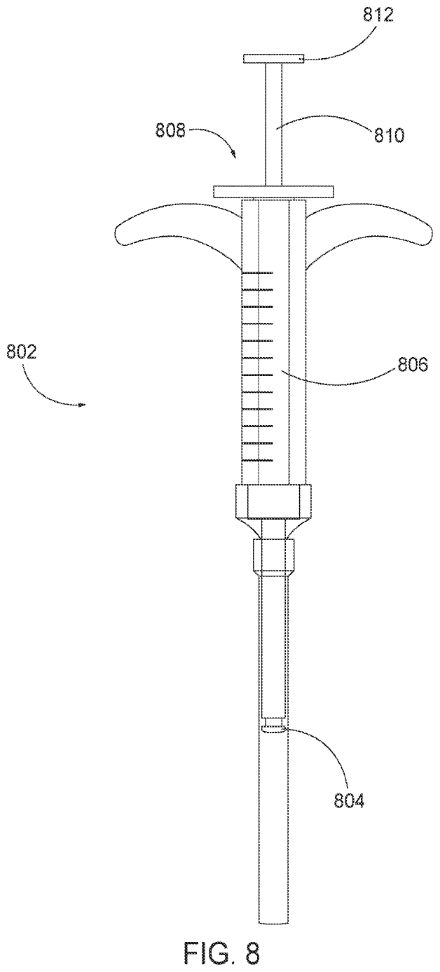

FIG. 8 shows a graft injection device 802 featuring a secondary graft plunge member 804 positioned distal to a barrel or tubular body 806 and primary graft plunge member 808, the latter comprising a rod or shaft member 810 coupled, attached or formed integrally with a proximal handle or plunge cap 812. The secondary graft plunge member 804 may facilitate the expulsion of all or most graft material from within the body 806 of the device to minimize waste and ensure sufficient grafting coverage of a decorticated bony surface.

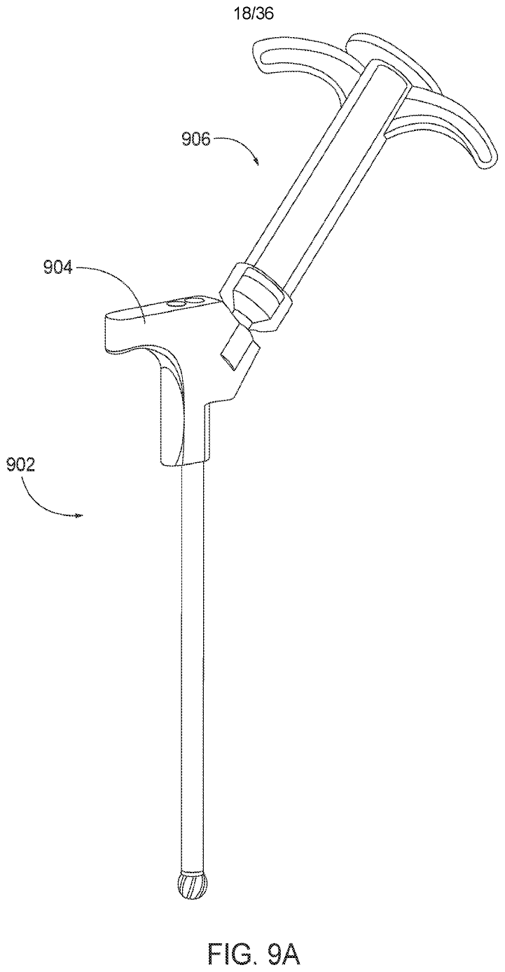

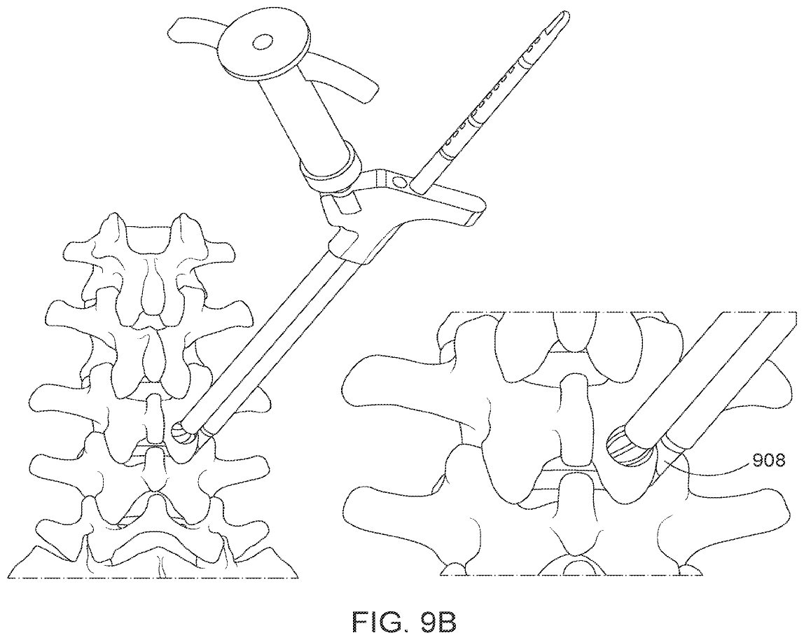

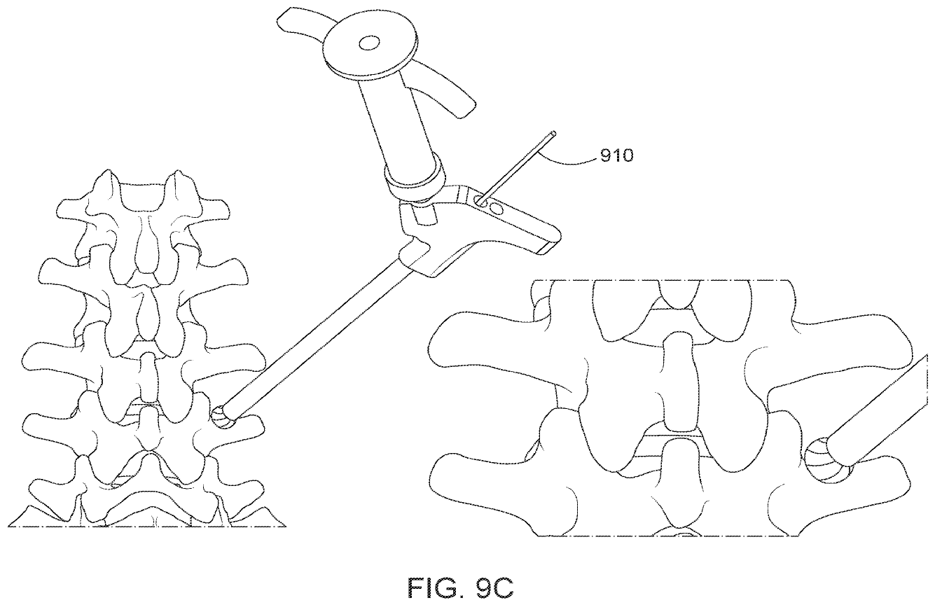

FIG. 9A shows a decortication device 902 that includes an angled proximal handle member 904 coupled to a graft injection device 906. This configuration may advantageously have a lower profile than other embodiments disclosed herein and existing in the art, which may facilitate tissue access and eliminate the need for an access portal. The decortication device 902 may be placed over an access chisel 908 (FIG. 9B) or a pedicle k-wire 910 (FIG. 9C).

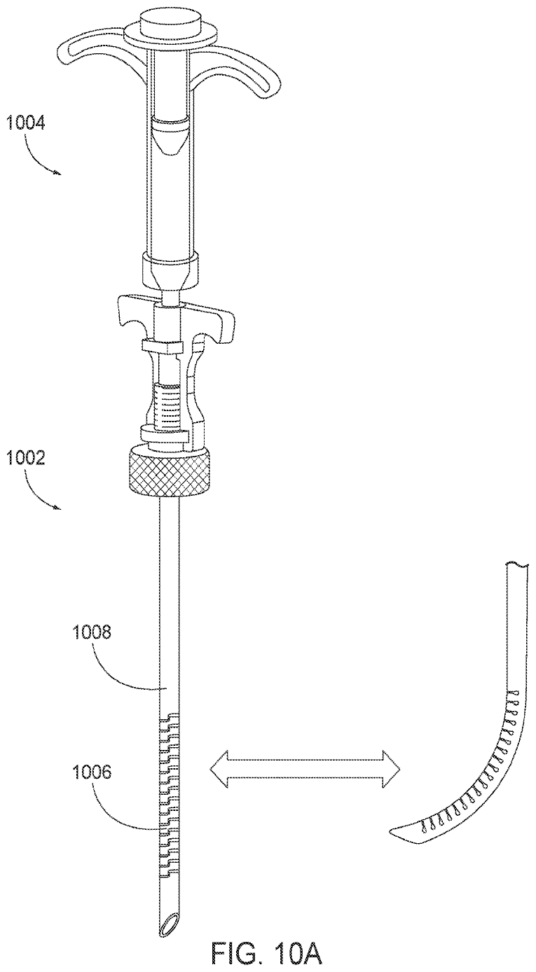

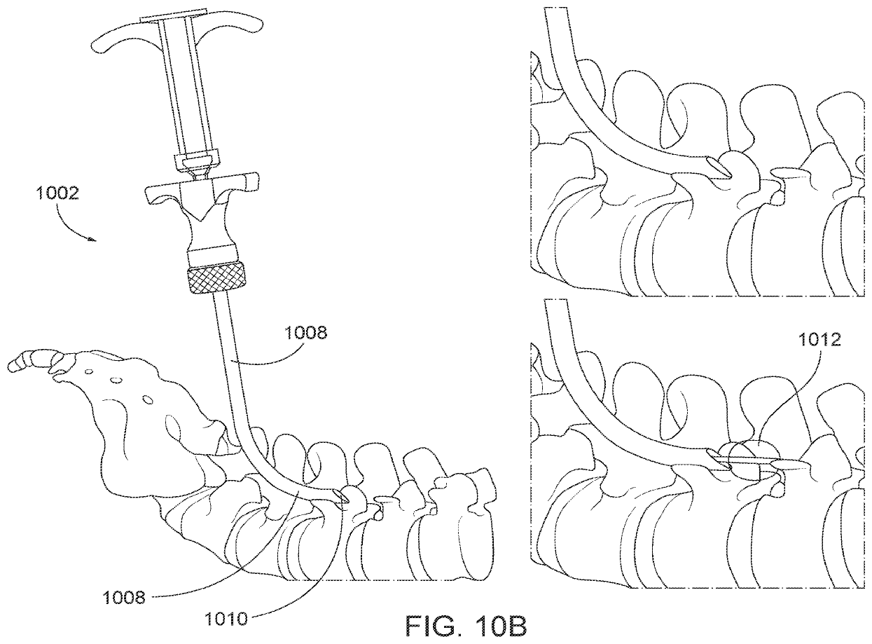

FIG. 10A shows an embodiment of a steerable graft delivery device 1002 coupled to a graft injection device 1004. At least a distal portion 1006 of a tubular portion 1008 of the graft delivery device 1002 may be configured to curve or contort as necessary to navigate complex anatomical locations, as further shown in FIG. 10B, where graft material may be selectively deposited through a distal opening 1010 of the tubular portion 1008. In operation, a pedicle screw tissue incision may be used to gain posterior access, after which the tubular portion 1008 may be navigated across the transverse process until a target site is reached, where the graft material is deposited. In some examples, an inflatable dilation device 1012 (e.g., a balloon) may be extended through the tubular portion 1008 of the device 1002 to dilate tissue prior to graft delivery.

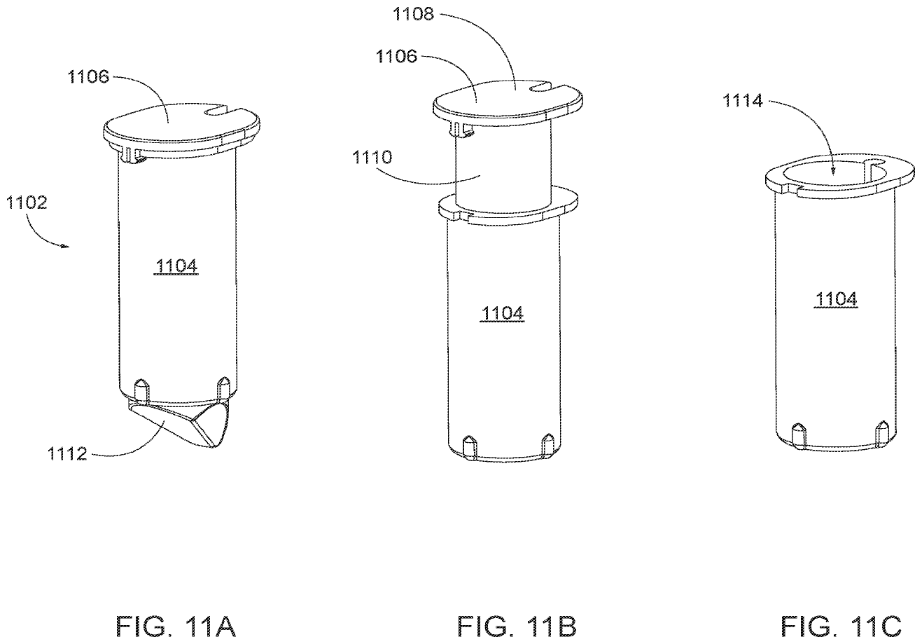

FIGS. 11A and 11B show an embodiment of an access portal device 1102 having an approximately cylindrical body 1104 coupled to a dilation device 1106, the latter featuring a cap 1108 and a shaft portion 1110 culminating in a tapered distal end portion 1112 configured to be urged through or between tissues and spread apart the tissues to enable clear access to the adjacent or underlying bony surfaces. As shown in FIG. 11C, the body 1104 of the access portal device 1102 may define a lumen 1114 configured to receive the shaft portion 1110 of the dilation device 1106 and other instruments used during a spinal fusion procedure, including a decortication and/or graft delivery device.

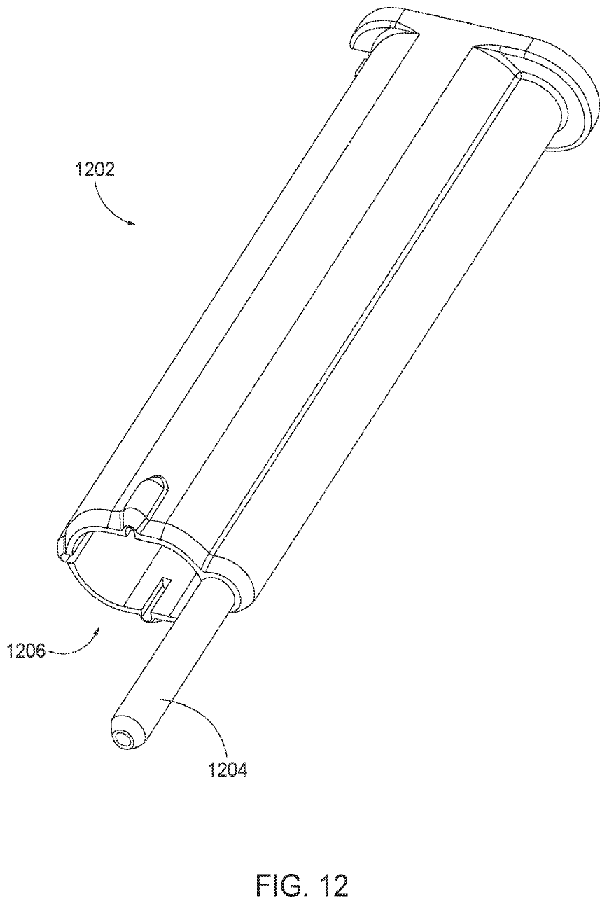

The access portal device 1202 shown in FIG. 12 includes an integrated pedicle stylet 1204 extending from a distal end 1206 of the device. The pedicle stylet 1204 may be inserted over a k-wire and into a pedicle pilot hole, where it may provide additional rigidity for improved device anchoring.

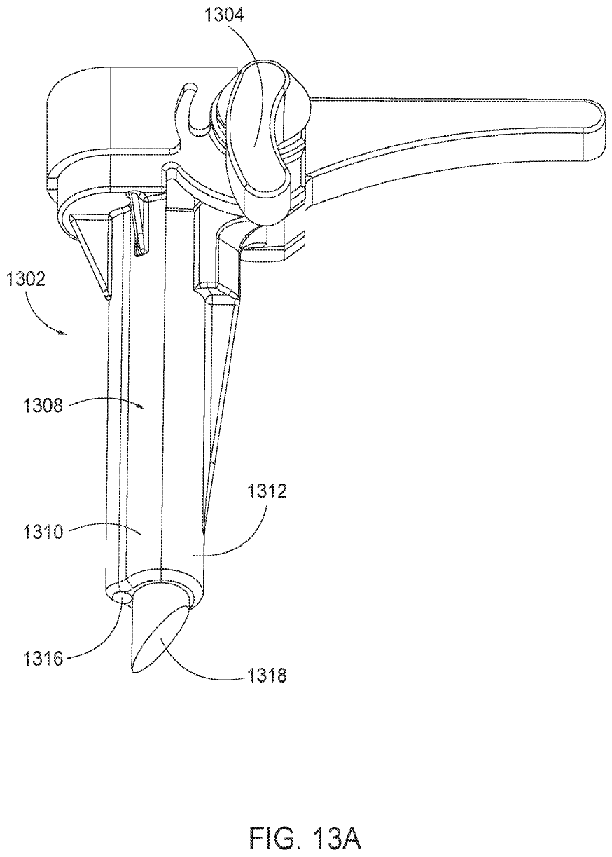

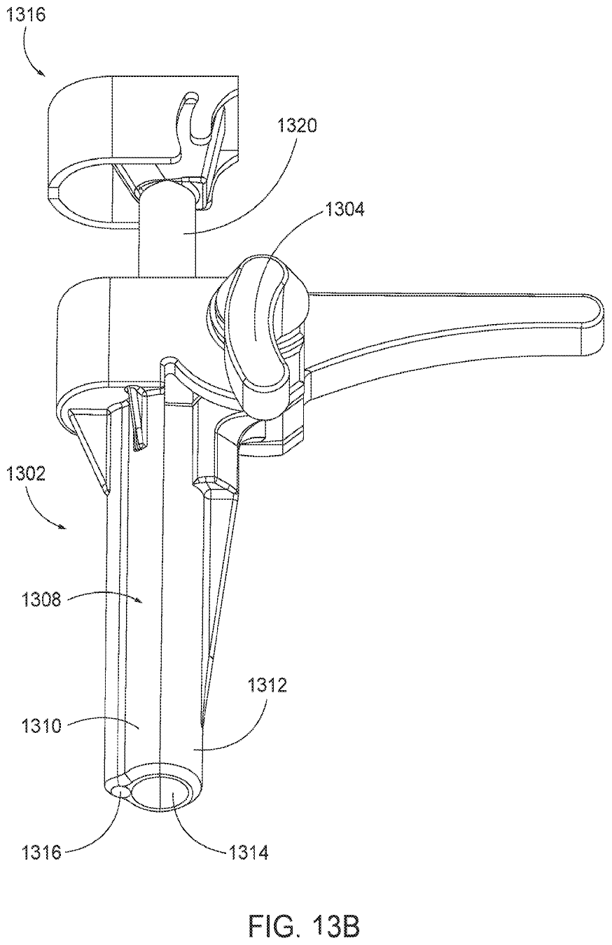

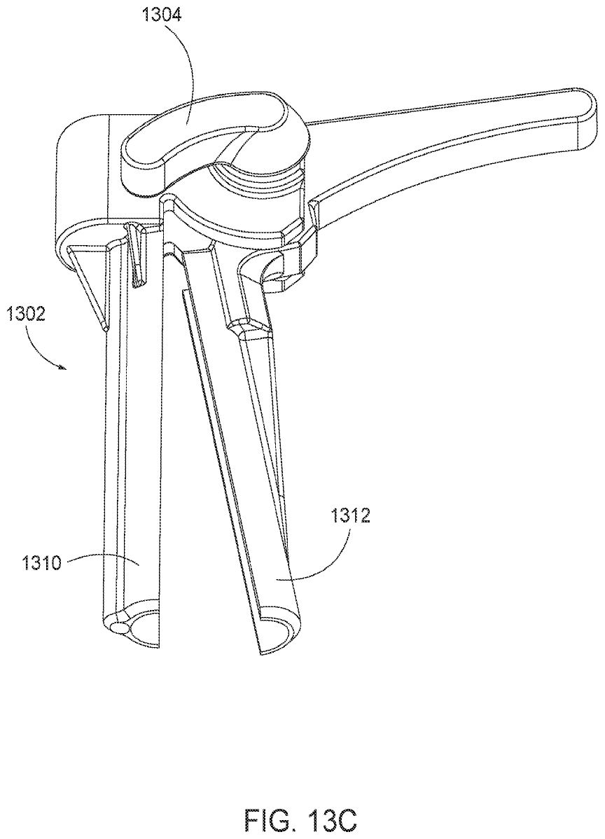

FIGS. 13A-13C depict various configurations of a retractor portal device 1302 comprising a latch member 1304, a first lumen 1306, and a tubular portion 1308 comprising a first side wall 1310 and a second side wall 1312 together defining a second lumen 1314. A dilation device 1316 defining an angled distal end portion 1318 and a shaft portion 1320 is shown coupled with the retractor portal device 1302. The retractor portal device 1302 is in an unlocked configuration in FIGS. 13A and 13B, such that the shaft portion 1320 and distal end portion 1318 of the dilation device 1316 can be slid through the second lumen 1314. FIG. 13C depicts a locked configuration in which the opposing side walls 1310, 1312 are separated and the retractor portal device 1302 locked at a specific angle.

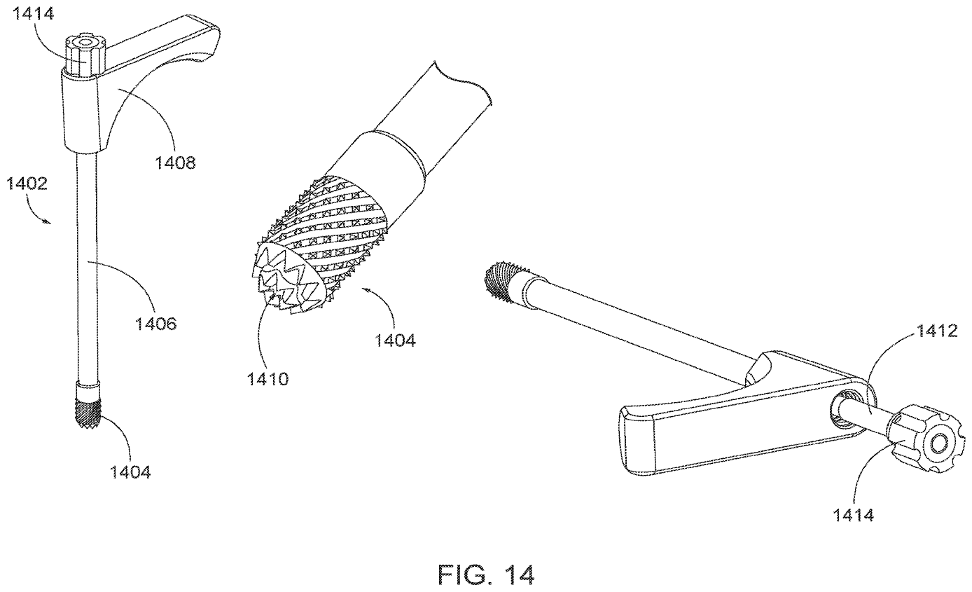

FIG. 14 shows a decortication device 1402 having a distal decortication tip 1404, a tubular portion 1406, and a proximal handle member 1408. The decortication tip 1404 comprises an approximately pinecone-shaped tip and an inner lumen 1410 configured for slidably coupling to a k-wire. As further shown, the decortication device 1402 may include or be coupled with a removable inner stylet 1412 through which graft material may be injected for deposition at a targeted bony surface. A proximal knob 1414 may be included to secure/release the stylet 1412 to the proximal handle member 1408.

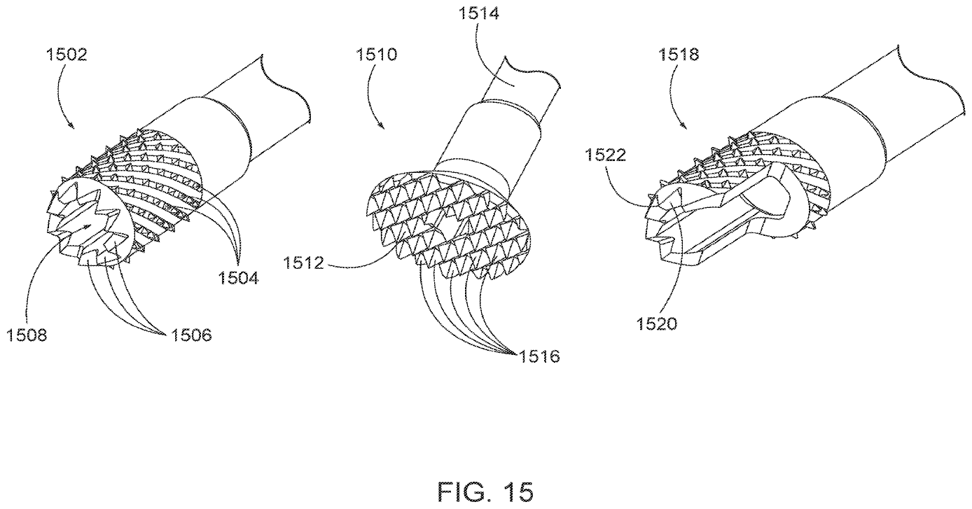

FIG. 15 depicts additional embodiments of decortication tips in accordance with various embodiments described herein. Decortication tip 1502 is similar to decortication tip 1404, having an approximately pinecone shape defined by a spiral pattern of serrations or teeth 1504. The distal-most ends of the tip 1502 include distally extending, jagged ridges 1506 surrounding an inner lumen 1508. Decortication tip 1510 includes a distal surface 1512 arranged substantially perpendicular to the tubular portion 1514 of the coupled decortication device. The distal surface 1512 is covered in jagged ridges, serrations and/or teeth 1516. Decortication tip 1518 includes a chamfered distal end 1520 with a plurality of serrated teeth 1522. The chamfered distal end 1520 defines the distal-most edge of the distal overhang defining the tip 1518.

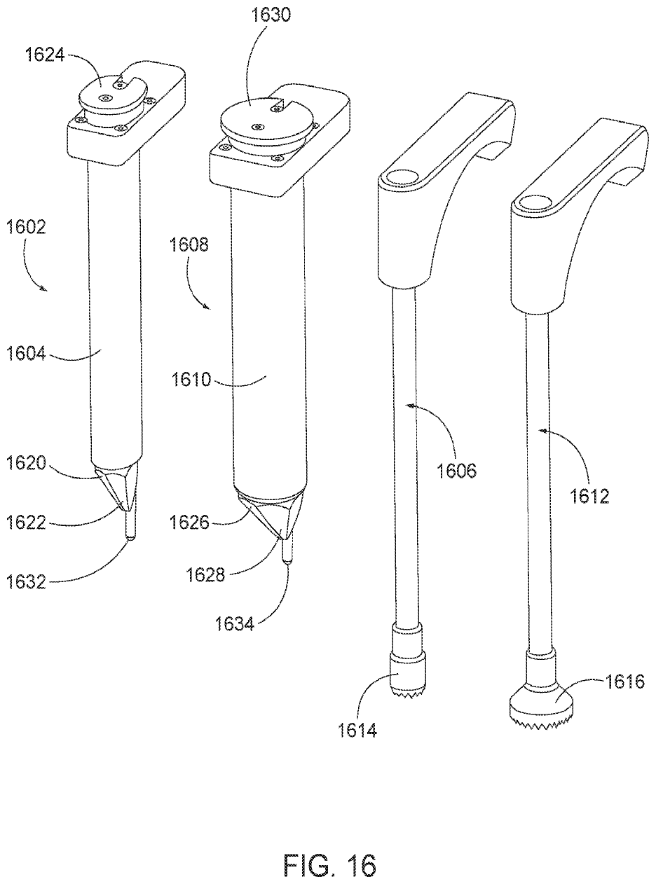

In some examples, one or more access portal devices may be configured to couple with one or more specific decortication device(s). The access portal device 1602 shown in FIG. 16, for instance, includes a body 1604 defining an inner surgical device lumen (e.g., lumen 206) sized and configured to receive decortication device 1606. By contrast, access portal device 1608 includes a body 1610 defining an inner surgical device lumen compatible with both decortication device 1606 and decortication device 1612.

The cross-sectional size and shape of the inner surgical device lumen of each access portal device (oriented transverse to the longitudinal access of the corresponding body 1604, 1610) together with the size and configuration of the decortication tip of each decortication device determines the device compatibility. In the example shown, the inner surgical device lumen and body 1604 of access portal device 1602 has a substantially oval cross-sectional shape configured to accommodate the distal decortication tip 1614 of decortication device 1606, but not the distal decortication tip 1616 of decortication device 1612. The inner surgical device lumen and body 1610 of access portal device 1608 has a substantially circular cross-sectional shape configured to accommodate decortication tip 1614 and the larger, more circular decortication tip 1616. Accordingly, embodiments of the access portal devices disclosed herein may be configured for coupling with one or more specifically configured decortication devices, and vice versa. Access portal devices configured to receive multiple different decortication devices may enable deployment and use of multiple decortication devices during a single procedure without changing access portal devices.

The size and shape of the access portal devices may also configure the devices to couple with different dilation devices. As further shown in FIG. 16, access portal device 1602 is configured to couple with dilation device 1620, which includes a tapered distal end portion 1622 and a proximal cap 1624. Access portal device 1608 is configured to couple with dilation device 1626, which includes a tapered distal end portion 1628 and a proximal cap 1630. The cross-sectional size of dilation device 1620 is smaller than the cross-sectional size of dilation device 1626.

As further shown, each access portal device 1602, 1608 may include an integrated pedicle stylet 1632, 1634, respectively, extending from the distal end of each device. Each stylet may be coaxial with a second inner lumen (or “side lumen”) of each access portal device, which may be configured to accommodate the passage of one or more anchoring devices, such as a k-wire, therethrough.

Spinal procedures implemented in accordance with the disclosed embodiments may involve the use of one or more access portal devices and one or more decortication devices. One non-limiting example of a posterolateral portal system or kit may include an oval access portal device, an oval dilation device, a round access portal device, a round dilation device, and multiple decortication devices, including for instance an approximately 11 mm decortication device and a 20 mm decortication device. Such a system or kit may be utilized to perform one or more procedures, and may enable the selection and swapping of different instruments “on the fly” as a procedure is performed.

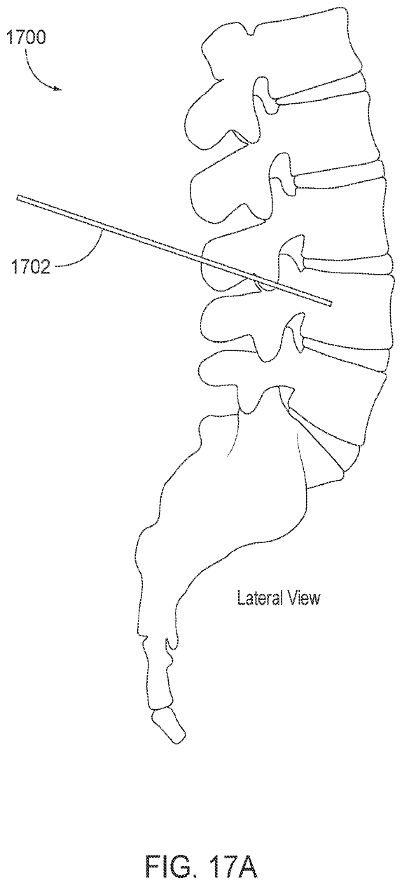

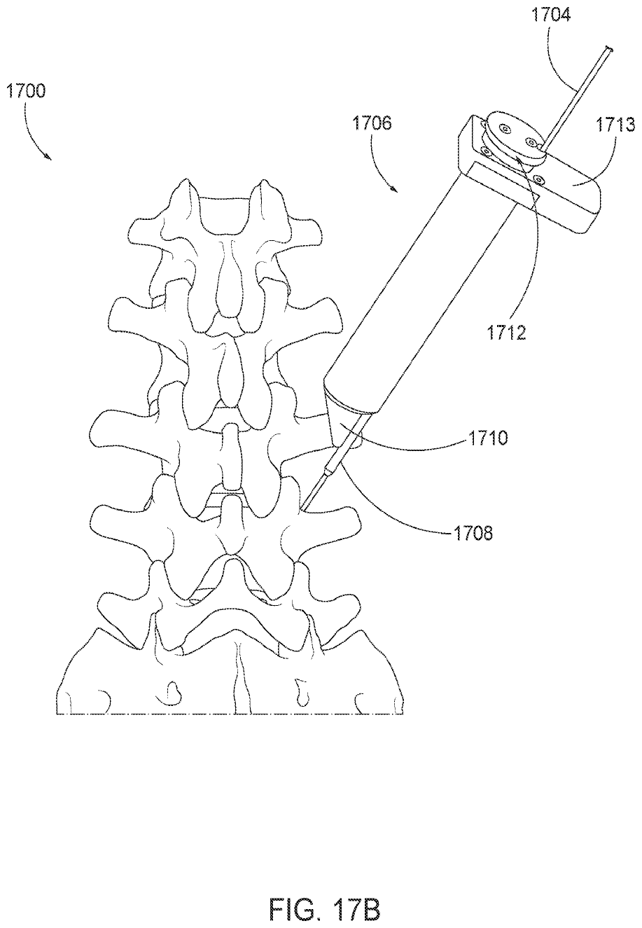

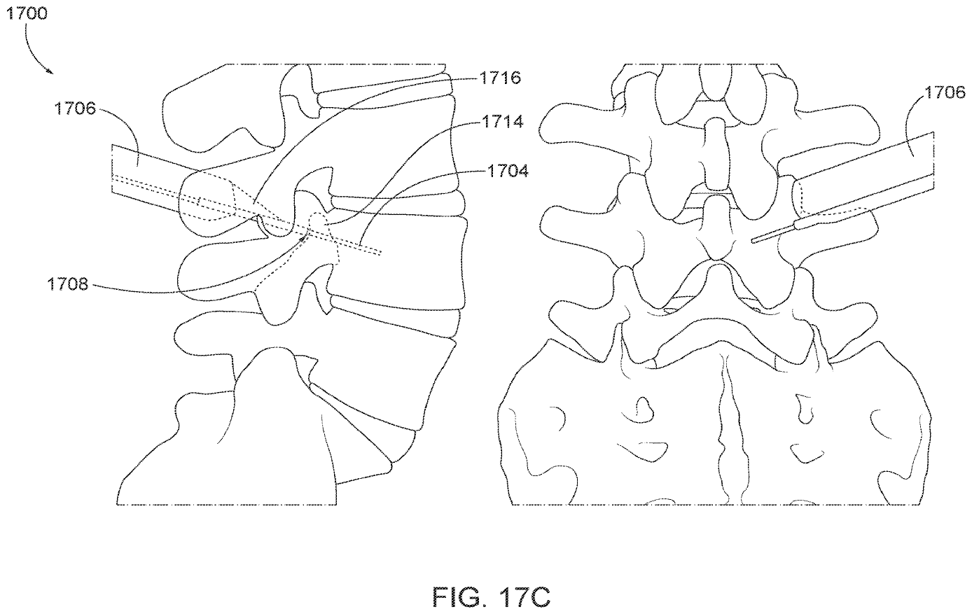

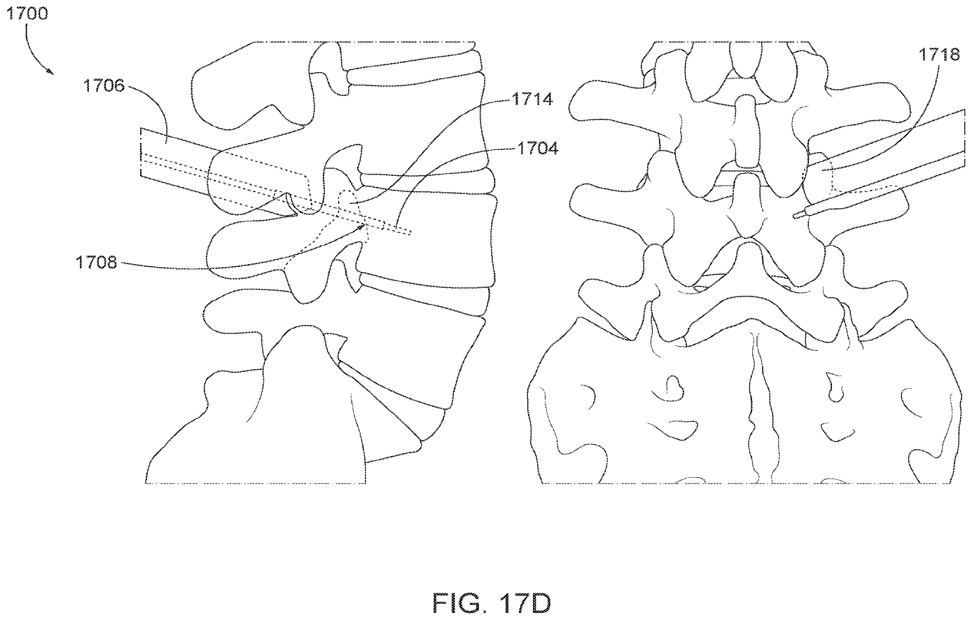

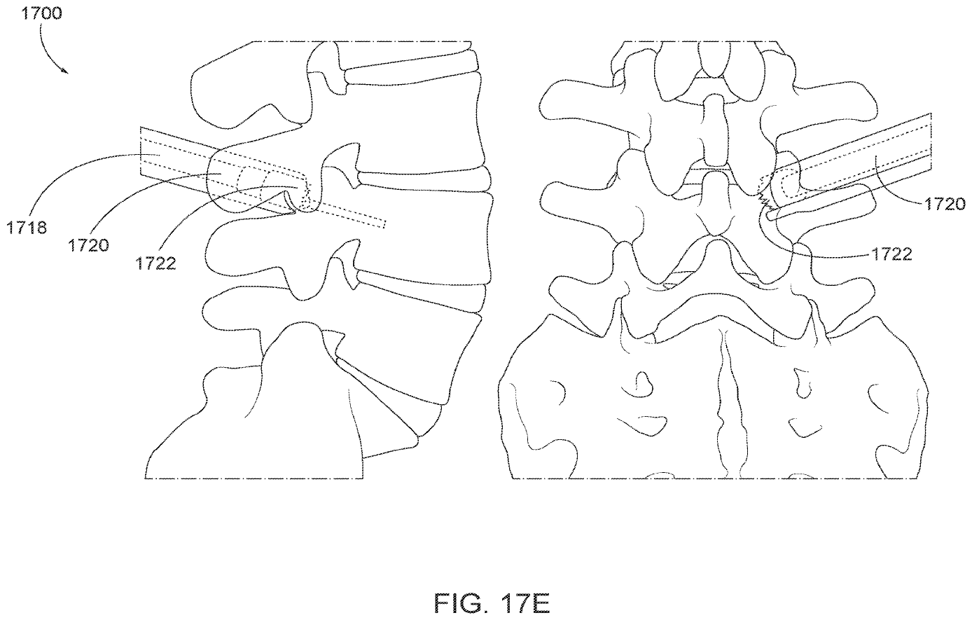

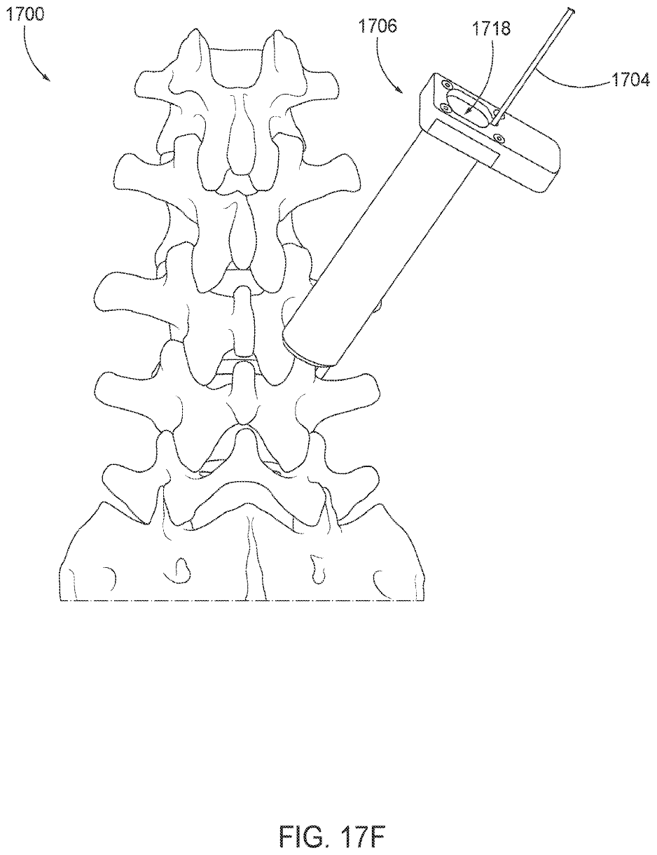

FIG. 17A is an illustration of an initial step of a spinal decortication and grafting procedure involving tissue distraction and surgical site access implemented in accordance with embodiments disclosed herein. A lateral view of a lumbar spinal region 1700 in a natural lordotic position is shown, which may be obtained by positioning the patient on a radiolucent table in a prone position. An elongate, hollow access needle 1702 (e.g., Jamshidi needle of various sizes, e.g., 11-gauge) may be used to gain access to the targeted lumbar pedicle, into which the access needle 1702 may create a pilot hole, as shown. Fluoroscopic guidance or navigation techniques can be used to access the pedicle. An anchoring device, such as a k-wire, may be extended distally through the access needle 1702 to the targeted pedicle, after which the access needle 1702 may be removed.

FIG. 17B illustrates a step of the procedure shortly after a vertical skin incision has been made at the entry point of the k-wire 1704, the incision sized to accommodate the insertion of a selected access portal device 1706. The size of the incision may be advantageously small relative to preexisting procedures, and may vary depending on the particular procedure and devices utilized. In one non-limiting example, the incision may be about 20-25 mm. A side lumen 1708 of the access portal device 1706, which may feature a distally extending tubular portion or stylet (e.g., 1632, 1634), may be slid over the k-wire 1704, thereby guiding the access portal device 1706 to the targeted boney surface. The access portal device 1706 may be coupled with a dilation device 1710 configured to facilitate passage of the access portal device 1706 through the tissue posterior to the targeted bone. Light malleting of the cap 1712 of the dilation device 1710 may facilitate its insertion together with the access portal device 1706. Malleting of the strike plate 1713 of the access portal device 1706 may facilitate deeper insertion of the portal device relative to the dilation device 1710 in the distal direction.

FIG. 17C shows a lateral view and an A-P view of the lumbar spinal region 1700. As shown, fluoroscopic imaging can be used to verify that the side lumen 1708 of the access portal device 1706 is indeed following the k-wire 1704 into the pedicle pilot hole created by the access needle 1702. The distal extension or stylet of the side lumen 1708 can be anchored into the pedicle 1714 after sliding distally along the k-wire 1704. The access portal device 1706 can be further advanced through the tissue via additional, light malleting of the cap 1712 of the dilation device 1710 until the distal end portion 1716 of the dilation device contacts the boney anatomical structure of the targeted surgical site. Lateral and A-P views may be used to confirm proper position of the devices.

FIG. 17D shows an additional step of the procedure, again from the lateral and A-P views, following removal of the dilation device 1710. The strike plate 1713 of the access portal device 1706 may be lightly malleted to advance the distal end of the access portal device 1706 beyond the distal end of the dilation device 1710 until the access portal device 1706 contacts the targeted bone. Once the location of the access portal device 1706 at the surgical site is confirmed, the access portal device 1706 may be moved, rotated or otherwise adjusted as necessary to position the device for decortication. The dilation device 1710 may be removed from the access portal device 1706, vacating the surgical device lumen 1718 defined by the portal device.

FIG. 17E illustrates a snapshot of the decortication process. A decortication device 1720 may be extended distally through the surgical device lumen 1718 of the access portal device 1706 until its textured decortication tip 1722 reaches the targeted bone. Tactilely contacting the targeted bone prior to decorticating may be important to avoid causing unwanted damage and/or incorrect positioning of the instruments. In some examples, the k-wire 1704 may be removed from the side lumen 1708 and reinserted one or more times to prevent instrument interference with the decortication device 1720 during its insertion and/or adjustment. A Bovie and suction procedure may be implemented to directly visualize the surgical site, if necessary. The targeted bone may be decorticated by manipulating the decortication tip 1722 in the manner described herein until the precise, complete decortication is confirmed in the lateral and A-P views. The access portal device 1706 may be rotated and repositioned as necessary to additional surgical sites, where the decortication process may be repeated.

In embodiments where grafting is required, the graft material may be deposited down through the inner surgical device lumen 1718 of the access portal device 1706 and tamped using the decortication device 1720. If the graft material is delivered to multiple anatomical locations, the access portal device 1706 may be pulled posteriorly and repositioned to the next desired surgical location, as represented in FIG. 17F. Posterior retraction of the access portal device 1706 may prevent or reduce the likelihood of “windshield wiping” the graft material away from a current surgical site. The k-wire 1704 may be reinserted down the side lumen 1708 of the access portal device 1706 to relocate the targeted pedicle if the access portal device is pulled back too far in the posterior direction.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. A spinal decortication and grafting system, comprising:
an access portal device defining a first lumen and a second lumen, each lumen extending longitudinally through a length of the access portal device, wherein the first lumen is configured to receive an anchoring device and the second lumen is configured to receive one or more surgical instruments;
a decortication device comprising a tubular portion and a distal decortication member; and
a graft injection device configured to couple with and push grafting material through the tubular portion of the decortication device,
wherein the tubular portion of the decortication device is configured to extend into and through the second lumen of the access portal device until the decortication member is positioned at a targeted bony surface.

2. The system of claim 1, wherein the anchoring device comprises a k-wire.

3. The system of claim 1, wherein the anchoring device comprises an access chisel.

4. The system of claim 1, wherein cross-sectional dimensions of the second lumen are greater than cross-sectional dimensions of the first lumen.

5. The system of claim 1, wherein the graft injection device comprises a plunge member and a tubular portion defining an inner lumen, the plunge member configured to urge the graft material through the inner lumen in response to manual actuation.

6. The system of claim 1, wherein the decortication device further comprises a handle attached to a proximal end of the tubular portion.

7. The system of claim 6, wherein the handle defines a coupling component configured to couple to a distal end portion of the graft injection device.

8. The system of claim 7, wherein the coupling component comprises a luer-thread fitting.

9. The system of claim 1, wherein the distal decortication member comprises a plurality of serrated teeth or ridges configured to decorticate the targeted bony surface via rotation or scraping.

10. The system of claim 1, wherein the distal decortication member comprises a substantially spherical burr covered in wavy ridges, serrated teeth or protrusions.

11. The system of claim 1, further comprising a dilation device configured to extend through the second lumen, the dilation device comprising a tapered distal portion configured to spread apart tissue at or near the targeted bony surface.

12. The system of claim 1, wherein the access portal device is configured to accommodate differently sized decortication devices having differently configured distal decortication members.

13. The system of claim 1, wherein the targeted bony surface is a feature of a lumbar vertebrae.

14. A decortication device for spinal fusion procedures, the decortication device comprising:
an elongate tubular portion configured to receive and facilitate passage of a graft material therethrough;
a decortication member attached to a distal end of the elongate tubular portion and configured to decorticate a bony surface; and
a handle member attached to a proximal end of the elongate tubular portion and configured to rotate the decortication member in response to manual actuation.

15. The decortication device of claim 14, wherein the handle member defines a coupling component configured to couple to a graft injection device and receive the graft material ejected therefrom and the coupling component comprises a luer-thread fitting.

16. (canceled)

17. The decortication device of claim 14, wherein the decortication member comprises a plurality of serrated teeth or ridges configured to decorticate the bony surface via rotation or scraping.

18. The decortication device of claim 14, further comprising an adjustable depth stop on the tubular portion configured to prevent over-extension of the decortication member beyond the bony surface.

19. The decortication device of claim 14, wherein the handle member comprises an angled body such that a coupling component defined by the handle member is angled with respect to a longitudinal axis of the elongate tubular portion.

20. The decortication device of claim 14, wherein a distal portion of the elongate tubular portion is configured to curve or contort away from a longitudinal axis of the elongate tubular portion.

21. (canceled)

22. The decortication device of claim 14, wherein the bony surface is a feature of a lumbar vertebrae

23.-24. (canceled)
